(19) United States
(12) Patent Application Publication
(10) Pub. No.: US 2023/0180743 A1
(43) Pub. Date: Jun. 15, 2023

(54) PROCESS FOR COATING AN OBJECT, USE OF NANOPARTICLES AND AN OBJECT WITH A COATED SURFACE

(71) Applicant: Grohe AG, Hemer (DE)

(72) Inventors: Torsten MEIER, Duesseldorf (DE); Matthias NAUSS, Bochum (DE)

(73) Assignee: Grohe AG, Hemer (DE)

(21) Appl. No.: 17/924,021

(22) PCT Filed: May 5, 2021
(86) PCT No.: PCT/EP2021/061836

§ 371 Date: Dec. 5, 2022

(30) Foreign Application Priority Data
May 9, 2020 (DE) 10 2020 112 602.6
Aug. 28, 2020 (DE) 10 2020 122 574.1
Sep. 9, 2020 (DE) 10 2020 123 494.5

(51) Int. Cl. A01N 25/10; A61L 2/232; A61L 2/238; A01N 59/20; A01N 59/16; A01P 1/00

(52) U.S. Cl. CPC A01N 25/10; A61L 2/232; A61L 2/238; A01N 59/20; A01N 59/16; A01P 1/00; A61L 2101/26

(57) ABSTRACT

A method for coating an object for equipping a building (1), in particular building hardware, at least having the steps outlined below: a) providing the object (1); and b) coating a surface (2) of the object (1) with a layer (3), wherein the coating is performed using a PVD process, in which antimicrobial or antiviral nanoparticles (4) are at least partially embedded into the layer (3). In addition, a use of nanoparticles, in particular for a PVD process for coating a surface (2) of an object (1), and an object (1) having a coated surface (2) are disclosed.

This invention relates to a method for coating an object, a use of nanoparticles, in particular for coating the object, and an object having a coated surface. The object may be, in particular, an object for equipping a building or a construction fitting or a sanitary item.

Windows, doors and gates and similar devices for opening or closing passages in buildings, e.g., for the passage of people and/or vehicles or for the exchange of air, regularly have operating elements, such as actuating levers, actuating switches and/or actuating buttons, which are contacted when used by a user. This can cause bacteria and/or viruses to accumulate on the operating elements, which can cause illness. To prevent the transfer of bacteria and/or viruses to different users, the operating elements often require extensive disinfection. This is especially the case when they are installed in hospitals or residential facilities for seniors.

The object according to the invention may also be a sanitary item. The sanitary item can in particular be a sanitary faucet by means of which in particular a liquid can be dispensed at a washbasin, shower, bathtub and/or WC.

Such sanitary items regularly have operating elements, such as actuating levers, actuating switches and/or actuating buttons, which are contacted when used by a user. This can cause bacteria and/or viruses to accumulate on the operating elements, which can cause illness. To prevent the transfer of bacteria and/or viruses to different users, the sanitary items often require extensive disinfection. This is especially the case when they are installed in hospitals or residential facilities for seniors.

Therefore, the invention addresses the problem of solving at least a part of the issues described with reference to the prior art and, in particular, of providing a method for coating an object, which can be used to at least reduce the effort required for disinfecting the object. In addition, a use of a PVD process for coating a surface of an object is to be disclosed, whereby the effort required to disinfect the object can at least be reduced. Furthermore, an object is to be specified, the effort to disinfect of which is at least reduced. The aforementioned object may be an object for equipping the structure of a building or a fitting. The object may be connected to the building or the structure of the building. For instance, the building may be a residential building, an office building, or a factory building.

These problems are solved by a method, a usage and an object having the features of the independent claims. Further advantageous embodiments of the invention are specified in the dependent claims. It will be appreciated that the features listed individually in the dependent claims may be combined in any technologically useful manner and define further embodiments of the invention. In addition, the features indicated in the claims are further specified and explained in the description, wherein further preferred embodiments of the invention are illustrated.

A method for coating an object used for equipping a structure of a building having at least the steps listed below contributes to solving the problem:
a) providing the object to equip the structure of a building, such as the sanitary item; and
b) coating a surface of the object with a layer, wherein the coating is performed using a PVD process, in which antimicrobial or antiviral nanoparticles are at least partially infused into the layer.

The object for equipping the structure of a building may be a fitting such as a construction fitting, for instance door hardware or window hardware. The window hardware may be, in particular, a window handle or a window stay or a window hinge. The door hardware can be a door handle or a door hinge. Further, the object for equipping the structure of a building may also be an object for equipping a driveway or a property boundary (e.g., wall, fence) of the assigned structure of the building, or an operating element for a doorbell or a light switch or a mailbox or the like. The object may be an object that can be touched by a user. The term structure of the building may refer to, for instance, the walls or partitions or parts of the building at least substantially secured thereto or to the ground, such as doors or windows.

The sanitary item is, in particular, a technical component used for water supply and/or wastewater disposal. In particular, the sanitary item may be used to supply water on demand to a sink, wash basin, shower, bathtub, and/or toilet. Furthermore, the sanitary item may be a housing or an actuating element, such as an actuating lever, actuating knob, actuating plate and/or rotating element, for such a technical component. The sanitary item can at least partially be made of plastic or metal, such as zinc die casting.

The object, e.g., the surface thereof, may comprise or consist of a metal.

According to optional embodiments of the invention, the object may, on the other hand, also be an object for equipping the structure of a building that is anything but a sanitary item or a sanitary faucet or a water-conveying object.

The object, e.g., at least the surface thereof, may comprise or consist of a metal such as, for instance, brass, steel, bronze, aluminum, magnesium, etc.

The object may be door hardware such as a door handle or a door hinge. A door handle is in particular a technical component that is used to open or close a lock of a door and/or to move a door relative to a door frame. Furthermore, the door handle may be an actuating element, such as an actuating lever, actuating knob, door knob, door handle, actuating plate, and/or rotary element. The door handle may be at least partially made of plastic or metal, such as brass, steel, bronze, aluminum, magnesium, etc. In particular, the door handle according to the invention is designed to be operated by the hand of a user. In accordance with embodiments of the invention, the term door handle may also include the assigned plate or collar disposed at the transition from the door to the door handle.

The window hardware may be a window handle or window hinges. The window handle is in particular a technical component that is used to open or close a lock of a window and/or to move a window relative to a window frame. Furthermore, the window handle may be an actuating element, such as an actuating lever, actuating knob, actuating plate, and/or rotary element. The window handle may be made at least partially or entirely of metal, such as brass, steel, bronze, aluminum, magnesium, etc. In particular, the window handle according to the invention is designed to be operated by the hand of a user. In accordance with embodiments of the invention, the term window handle may also include the assigned plate or collar disposed at the transition from the window to the window handle. The window hardware may further be a window stay or a window frame.

In step a), the object is provided to equip the structure of a building. In particular, this may include placing the object in a coating chamber of a reactor adapted to perform a physical vapor deposition (“PVD”) process. The PVD process is a (thin film) coating process using physical vapor deposition. The object can be disposed in the coating chamber, for instance, on or at a support or rotating element, such as a turntable or the like, by means of which the object can be rotated during coating. This can form a uniform layer on a surface of the object.

In step b), the surface of the object is coated using the PVD process, in which a layer is formed on a surface of the object. The layer can be formed from a plurality of overlapping individual layers and/or have a layer thickness of 1nm (nanometer) to 20 μm (micrometer). In particular, a vacuum or controlled atmosphere can be created in the coating chamber of the reactor prior to the actual coating process. The material(s), of which the coating is to be made, is/are provided during the coating process, for instance, in the form of gases, vapors, and/or particles within the coating chamber of the reactor. In this process, the gases, vapors and/or particles come into contact with the surface of the object to be coated and are deposited thereon. The material(s) may be, for instance, pure or alloyed metals, nitrides, carbonitrides, and/or oxides.

During the coating of the surface, antimicrobial and/or antiviral nanoparticles are embedded in the formed layer for them to be able to render pathogens, such as bacteria or viruses, that get onto the layer when the object is used, harmless. For this purpose, a mass fraction of nanoparticles in the layer can be, for instance, 1% to 80%. The nanoparticles can be provided and/or generated directly in the coating chamber of the reactor or in an additional container. For this purpose, the coating chamber can be connected to the additional container via a channel, for instance. The channel can have a valve, which can be used to control a feed of the nanoparticles into the coating chamber. When the layer is formed on the surface of the object, the nanoparticles come into contact with the layer and are at least partially embedded in the layer in that way. As a result, the nanoparticles are attached to the surface of the object to be coated.

The nanoparticles can be generated, for instance, by evaporation, ablation, plasma formation or exfoliation of a solid, liquid or gaseous starting material. The starting material may contain the nanoparticles or be made of the same material from which the nanoparticles are to be formed. Vaporization of the starting material can be accomplished, for instance, by heating or by techniques known as magnetron sputtering. Evaporation of the starting material can alternatively be performed by bombarding with ion or electron beams (IB-PVD process or EB-PVD process) or using a pulsed high-power laser beam (laser beam evaporation). After the nanoparticles are generated, they can be transported to the coating chamber of the reactor. This can be done, for instance, using the help of gravity, a pressure difference, and/or by a flow of a carrier gas or liquid. Such a stream of nanoparticles and carrier gas or liquid can also be delivered to the reactor by ion or plasma sources or various delivery systems. Alternatively, the nanoparticles can be generated directly in the reactor. In this case, the nanoparticles or starting material are provided in the coating chamber of the reactor and/or on the support or rotating element for the object.

Owing to the antimicrobial and/or antiviral effect of the nanoparticles, disinfection of the coated surface of the object is not necessary or the effort required to disinfect the object can at least be reduced.

The nanoparticles can have a diameter of 1 nm to 10 nm.

The nanoparticles may at least partially consist of silver. In particular, the silver may be colloidal silver.

The nanoparticles may consist at least partially of copper.

The nanoparticles may consist at least in part of zinc or zinc oxide.

The nanoparticles may consist of an alloy comprising at least silver, copper, zinc and/or zinc oxide.

The layer may at least partially form a contact surface of the object. In particular, the contact surface is a surface that can be or is contacted when the object is used by a user.

The object may be an actuator for a window, door, gate, or similar obstruction for controlling the access to a building or property. In particular, the door handle may be a component of a door or gate.

According to another aspect, a use of a PVD process for coating a surface of an object with a layer is also proposed, wherein antimicrobial or antiviral nanoparticles are at least partially infused in the layer.

Further proposed is the use of nanoparticles comprising at least silver, copper, zinc, zinc oxide, or alloys thereof to produce at least one antimicrobial or antiviral layer of a surface of an object.

According to yet a further aspect, an object having a surface coated with a layer in which antimicrobial or antiviral nanoparticles are at least partially embedded is also proposed.

For further details on the use or the object, reference is made in full to the description of the process.

The invention and the technical environment are explained in more detail below with reference to the figures. It should be noted that the figures show a particularly preferred embodiment variant of the invention, but the invention is not limited thereto. The same reference numerals are used for the same components in the figures. Schematically:

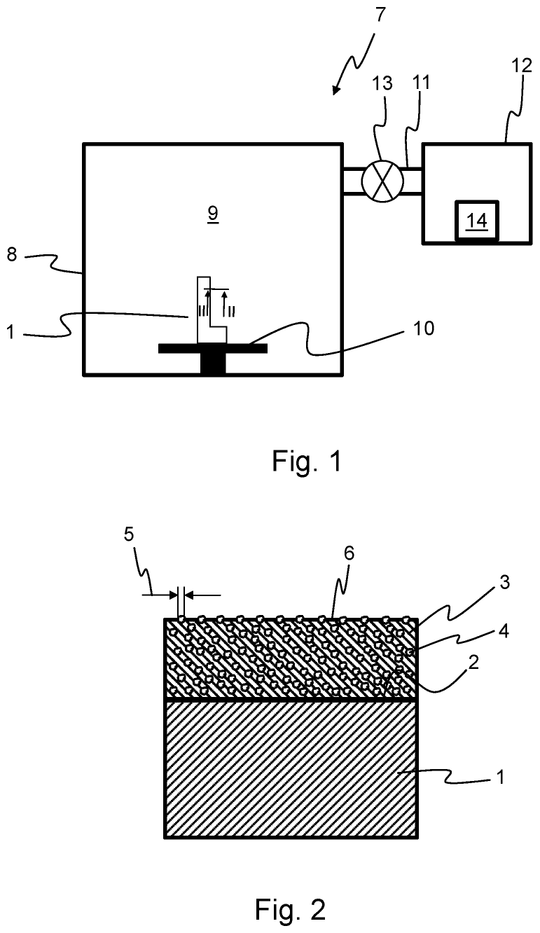

FIG. 1 shows a device for performing the method; and

FIG. 2 shows a door handle according to one embodiment of the invention in a (partially) sectional view.

FIG. 1 shows a device 7 for performing a process for coating an object for equipping the structure of a building 1, wherein in the embodiment of FIG. 1 the object is a door handle. Notwithstanding, the process described below can also be used to coat other objects used to equip the structure of a building. The device 7 comprises a reactor 8 having a coating chamber 9, in which the door handle 1 is rotatably provided on a rotating element 10, for instance in the manner of a turntable. The door handle 1 in this case is a door handle that is set up to unlock a door by rotating the door handle relative to the door. A PVD process is used to coat a surface 2 of the door handle 1 shown in FIG. 2 with a layer 3 also shown in FIG. 2. For this purpose, a vacuum or a controlled atmosphere can be generated in the coating chamber 9 of the reactor 8. During the coating process, the material or materials of which the layer 3 is to be composed are made available, for instance, in the form of gases, vapors and/or particles in the coating chamber 9 of the reactor 8. These come into contact with the surface 2 of the door handle 1 to be coated and adhere to it. The layer 3 is thus formed by depositing the material or materials on the surface 2 of the door handle 1. The layer 3 may be of different types, thicknesses and densities and/or may be made of overlapping layers of the material(s).

The coating chamber 9 of the reactor 8 is connected to an additional container 12 via a channel 11. The container 12 is located outside the reactor 8 in this case. Alternatively, however, it can also be disposed in the coating chamber 9 of the reactor 8. The container 12 is used to provide the antimicrobial or antiviral nanoparticles 4 shown in FIG. 2. The connection of the coating chamber 9 of the reactor 8 to the container 12 can be interrupted by a valve 13 in the channel 11. In that way, the valve 13 can be used to either enable or completely or partially prevent a flow of the nanoparticles 4 into the coating chamber 9 of the reactor 8. During the coating of the surface 2 of the door handle 1, the valve 13 is at least partially opened for the nanoparticles 4 to enter the reactor 8 and come into contact with the layer 3. As a result, the nanoparticles 4 are at least partially embedded in the layer 3 and consequently attached to the surface 2 to be coated.

For instance, the nanoparticles 4 can be generated in the container 12. This can be done, for instance, by evaporation, ablation, plasma formation, or exfoliation of a solid, liquid, or gaseous starting material 14. After the nanoparticles 4 have been generated, they are conveyed into the reactor 8 via the channel 11 while the valve 13 is at least partially open. This can be done, for instance, using gravity, a pressure difference, and/or by a flow of a carrier gas or carrier liquid.

FIG. 2 shows a sectional representation of the door handle 1 along the line of intersection II-II shown in FIG. 1. In particular, the surface 2 of the door handle 1 coated with the layer 3 can be seen here. The nanoparticles 4 are at least partially embedded in the layer 3. In this way, they can act antimicrobially and/or antivirally on a contact surface 6 of the door handle 1 formed by the layer 3. The nanoparticles 4 have a diameter 5, which can be, for instance, 1 nm to 10 nm.

This embodiment reduces the effort for disinfecting the door handle.

### LIST OF REFERENCE NUMERALS AND TERMS

1 object to equip the structure of a building/door handle
2 surface
3 layer
4 nanoparticles
5 diameter
6 contact surface
7 device
8 reactor
9 coating chamber
10 rotary element
11 channel
12 container
13 valve
14 starting material

1. A method for coating an object for equipping a structure of a building (1), in particular hardware, at least comprising the steps listed below:
a) providing the object to equip the structure of the building (1); and
b) coating a surface (2) of the object (1) with a layer (3), wherein the coating is performed using a PVD process, in which antimicrobial or antiviral nanoparticles (4) are at least partially embedded into the layer (3).

2. The method according to claim 1, wherein the nanoparticles (4) have a diameter (5) of 1 nm to 10 nm.

3. The method according to claim 1, wherein the nanoparticles (4) at least partially consist of silver.

4. The method according to claim 1, wherein the nanoparticles (4) at least partially consist of copper.

5. The method according to claim 1, wherein the nanoparticles (4) at least partially consist of zinc or zinc oxide.

6. The method according to claim 1, wherein the nanoparticles (4) consist of an alloy comprising at least silver, copper, zinc or zinc oxide.

7. The method according to claim 1, wherein the layer (3) at least partially forms a contact surface (6) of the object (1) for contact by a user.

8. The method according to claim 1, wherein at least the surface (2) of the counterpart (1) consists of metal.

9. The method according to claim 1, wherein the object (1) is an actuating element for a light switch, a mailbox, a window, a gate, in particular a rolling gate, a revolving door, or a door pivotally mounted at a longitudinal end.

10. The method according to claim 1, wherein the object (1) is a sanitary item, in particular a sanitary faucet, an actuating element for the sanitary faucet, a toilet or an actuating element for the toilet.

11. Use of nanoparticles comprising at least silver, copper, zinc, zinc oxide or alloys thereof for producing at least one antimicrobial or antiviral layer (3) of a surface (2) of an object for equipping the structure of a building (1).

12. An object for equipping a building (1) comprising a surface (2) coated with a layer (3), in which antimicrobial or antiviral nanoparticles (4) are at least partially embedded.

13. The object (1) according to claim 12, wherein the object is manufactured using a method for coating an object for equipping a structure of a building (1), in particular hardware, at least comprising the steps listed below:
a) providing the object to equip the structure of the building (1); and
b) coating a surface (2) of the object (1) with a layer (3),
wherein the coating is performed using a PVD process, in which antimicrobial or antiviral nanoparticles (4) are at least partially embedded into the layer (3).
